(19) United States
(12) Patent Application Publication
(10) Pub. No.: US 2009/0324500 A1
(43) Pub. Date: Dec. 31, 2009

(54) SCREEN FOR INFLAMMATORY RESPONSE MODULATORS

(76) Inventors: Stephen Andrew Renshaw, South Yorkshire (GB); Philip William Ingham, South Yorkshire (GB); Moira Katherine Brigid Whyte, South Yorkshire (GB)

Correspondence Address: MYERS BIGEL SIBLEY & SAJOVEC, PO BOX 37428, RALEIGH, NC, 27627, US

(21) Appl. No.: 12/301,314

(22) PCT Filed: May 14, 2007
(86) PCT No.: PCT/GB07/01766

§ 371 Date: Apr. 23, 2009

(30) Foreign Application Priority Data
May 19, 2006 (GB) 0609946.9
Nov. 21, 2006 (GB) 0623120.3

(51) Int. Cl. A61K 49/00; C12Q 1/00; C12Q 1/02; C12Q 1/68; C12N 5/10

(52) U.S. Cl. 424/9.2; 435/40.5; 435/29; 435/6; 435/325

(57) ABSTRACT

The present invention relates to transgenic aquatic vertebrate organisms and methods of their use in screening for, or identifying agents that are useful in modulating the inflammatory response and particularly for identifying agents useful for treating inflammatory disorders.

### FIELD OF THE INVENTION

The present invention relates to methods of screening and/or identifying agents that are useful in modulating the inflammatory response and particularly but not exclusively for identifying agents useful for treating inflammatory disorders.

### BACKGROUND TO THE INVENTION

Early in the evolution of multi-cellular organisms, specific adaptations arose that enhanced survival in the presence of ongoing environmental challenges such as trauma or injury and infection. The inflammatory response is one such adaptation, and its success underpins the increasing complexity of all higher animals including man. Resolution of even the most intense inflammatory reactions with restoration of normal tissue function, e.g. lobar pneumonia, demonstrates how tight regulation of the inflammatory response has also evolved. Despite these tight controls on inflammation, much human suffering and premature mortality in the developed world can be attributed to diseases in which the inflammatory response fails to resolve adequately or promptly, and irreparable tissue damage occurs. Examples of such conditions in the lung include fibrotic lung disease, asthma and both adult and neonatal respiratory distress syndromes. Such conditions have parallels in almost any organ system and contribute to the burden of chronic organ failure, disability and death.

The mechanics of resolution of inflammation have largely been extrapolated from knowledge of the processes of initiation and propagation of inflammation. These include dissipation of inflammatory mediators, cessation of neutrophil and monocyte influx, restoration of normal microvascular permeability, control of inflammatory cell secretion and clearance of inflammatory cells and debris. Neutrophils, numerically the predominant cell of acute inflammatory response, undergo programmed cell death or apoptosis both in vitro and in vivo. Neutrophils are exquisitely sensitive to apoptosis, and show the highest rate of constitutive apoptosis of any cell type, but also show very significant modulation of apoptosis by cytokines and other survival factors. Apoptosis is thought to be fundamental to the resolution of inflammation, since it down-regulates the pro-inflammatory functions of granulocytes, including inhibition of release of toxic granule contents, which would perpetuate inflammation. Importantly, apoptotic neutrophils are specifically recognised by macrophages and subsequently phagocytosed and removed. In addition, macrophages that take up apoptotic neutrophils themselves exhibit changes in their pattern of cytokine secretion, which favour production of anti-inflammatory cytokines and resolution of inflammation. For these reasons the timely removal of neutrophils via apoptosis may be a pre-requisite for efficient resolution of inflammation with minimal tissue damage. The exact molecular controls of this process in vivo remain uncertain, although it appears that the interplay between external stimuli and an internal, genetic programme is important.

Screening for agents that are useful in treating inflammatory disorders with the traditionally used mammal models is laborious and costly. Embryonic stage zebrafish models have been investigated as a replacement to mammalian models (mice, rats) during the pre-clinical phase. Their perceived utility is mainly due to the conservation of several important biological characteristics during evolution. Consequently, the effects drug candidates on zebrafish can be very similar to those on humans, including physiological and molecular-biology characteristics. Moreover, zebrafish can be bred during the entire year, and a single mother fish lays a considerable number of eggs during spawning. The processes of organogenesis that take place very quickly can be easily studied in the transparent embryos developing outside the body. During the initial 4-5 days of its development zebrafish is feeding from the yolk sac, requiring therefore no external food. The extraordinarily rapid development of zebrafish embryos provides the possibility of rapid testing of drug candidates, because the fish embryo develops all of its important organs during two days and is within five days able to feed independently, becoming an independent animal susceptibly reacting to stimuli received from its environment. Tested drugs diffuse into the tissues of the embryo from the water surrounding the embryo. The transparency of the zebrafish embryos allows detection of the morphological and functional changes of the various organs affected by drugs, without any complicated surgical procedure required, contrary to mammals. For example necrosis can be immediately detected in the form of opalescence, and also the defects of circulatory system such as e.g. haemorrhage can be detected easily.

It is desirable to provide a rapid and inexpensive screen for the identification of candidate therapeutics useful in treating inflammatory disorders.

It is also desirable to identify agents/compounds that induce apoptosis specifically in granulocytes thereby eliminating unwanted granulocytes during inflammation and providing a treatment for inflammatory disease.

### STATEMENTS OF INVENTION

According to the broadest aspect of the invention there is provided a screening method for the identification of an agent that modulates an inflammatory response comprising:
i) assessing the amount of viable granulocytes in an aquatic vertebrate organism in the presence or absence of an applied inflammatory stimulus;
ii) exposing the said aquatic vertebrate organism to a candidate modulator agent and assessing the amount of viable granulocytes; and
iii) comparing the amount of viable, granulocytes obtained from step i) against those obtained from step ii), whereby a difference in values obtained in step i) and step ii) indicates whether the agent inhibits or enhances the inflammatory response.

That is to the method may be applicable to transgenically or otherwise genetically-modified aquatic vertebrate organisms. The screening method of the present invention provides a rapid, inexpensive and simple screen for candidate therapeutics that are capable of influencing the inflammatory response, the method being based on assessment of the amount or number of viable granulocytes in an aquatic vertebrate organism and a comparison against the amount or number of viable granulocytes when the organism is exposed to a candidate therapeutic. The aquatic vertebrate animal may also be optionally subjected to an inflammatory stimulus, in this way the method of the invention can be advantageously used to detect agents that inhibit or enhance the inflammatory response.

According to a first aspect of the invention there is provided a screening method for the identification of an agent that modulates an inflammatory response comprising:
i) assessing the amount of viable granulocytes in a transgenic aquatic vertebrate organism that expresses a reporter protein or product such that granulocyte apoptosis can be detected in vivo, in the presence or absence of an applied inflammatory stimulus;
ii) exposing the said transgenic aquatic vertebrate organism to a candidate modulator agent and assessing the amount of viable granulocytes; and
iii) comparing the amount of viable granulocytes obtained from step i) against those obtained from step ii), whereby a difference in values obtained in step i) and step ii) indicates whether the agent inhibits or enhances the inflammatory response.

It will be appreciated that steps i) and ii) may be performed in reverse order and that the strict ordering of these two steps is not intended to limit the scope of the application since it is the comparative values of viable granulocytes in the aquatic vertebrate organism in the presence or absence of an applied inflammatory stimulus when compared to the amount of viable granulocytes obtained when exposing the organism to a candidate modulator which allows for the identification of an inflammatory response modulator.

Reference herein to “an agent that modulates an inflammatory response” or “an inflammatory response modulator” is intended to include a natural or synthetic compound or agent which is capable of blocking or preventing initiation of the inflammatory response or is capable of reducing the severity and/or length of the response or of resolving the inflammatory response. In some instances, specific enhancement of the inflammatory response may be desirable, for example as part of a cancer therapeutic strategy. In these circumstances the modulator agent is capable of enhancing or prolonging the inflammatory response.

Reference herein to a “transgenic” organism is intended to include an organism whose sperm or egg contain genetic material originally derived from an organism other than the parents or in addition to the parental genetic material, the transgenic organism being an organism whose genome has been altered by the transfer of a gene or genes from another species

Reference herein to “an applied inflammatory stimulus” is intended to include mechanically damaging the organism by means of cutting or lasering a part of the external skin of the organism; exposing the organism to stress conditions such as hypoxia; disturbances of homeostatic balance such as changes in temperature or pH, infection; disease status such as malignancy; exposing the organism to a chemical insult such as a toxin or other injurious agent, for example tetanus toxin, diphtheria toxin or DPTP or a pro-inflammatory agent for example a chemokine or a bacterial product or synthetis homologue of a bacterial product, such as fMLP.

Reference herein to “viable” granulocyte is intended to include a healthy or normal or live or functionally competent or non-apoptotic granulocyte.

Reference herein to “amount of viable granulocytes” is a qualitative or quantitative assessment. In the instance where it is a qualitative assessment the comparison step of step iii) can be by eye in so far as the assessor can determine whether the staining level, intensity or distribution pattern of viable granulocytes is affected by the presence of the candidate modulator agent. In the instance where the assessment is quantitative the number or amount or distribution of viable granulocytes can be assessed by eye or can be calculated by, for example and without limitation digital image analysis. Details of the methodology for assessing the amount of viable granulocytes are discussed in more detail herein after.

The assessment of viable granulocytes can be assessed in either the whole organism or more preferably a part of the organism, for example and without limitation in a fin such as the tail fin or a portion thereof.

Preferably, the granulocyte is selected from the group comprising a mast cell (or mastocyte), eosinophil, basophil, neutrophil or heterophil. More preferably, the granulocyte is a neutrophil.

Preferably, the aquatic vertebrate organism is a fish or amphibian.

More preferably the fish is a zebrafish and the amphibian is a xenopus.

Preferably, the screening method is carried out in vivo and/or post mortem.

The screening method of the present invention is equally applicable to a live aquatic vertebrate organism or one which has been culled and may in one embodiment be performed on the same animal when both alive and dead.

In one embodiment of the invention, particularly in the instance of the screening method being performed in vivo, the aquatic vertebrate organism is a transgenic organism that expresses a granulocyte specific reporter protein or product that can be detected in vivo.

Reference herein to a “reporter protein” is any protein that can be specifically detected either directly or indirectly when expressed. Reporter proteins are useful for detecting and quantitating expression of expression sequences.

For example, the reporter product, expression of which is under the control of a granulocyte specific promoter such as the myeloperoxidase promoter, may be directly detected, removing the need for a substrate. Green fluorescent protein (GFP) has become one of the most commonly used examples of this category of reporter as a convenient read-out product for biological systems. This fluorescent protein was derived from the bioluminescent jellyfish Aequoria Victoria and several colour spectral variants of this reporter have been developed such as enhanced green fluorescent protein (eGFP), reef coral fluorescent protein (RCFP), cyan fluorescent protein (CFP), red fluorescent protein (RFP) and yellow fluorescent protein (YFP). Preferably, the method includes any suitable fluorescent read out reporter expression of which is under the control of a granulocyte specific promoter.

In one specific example of the methods of the present invention, the aquatic vertebrate organism is transgenicaly modified so as to express modified GFP under the control of the neutrophil specific myeloperoxidase promoter. Preferably the method of assessing the amount of viable granulocytes is achieved in vivo by counting or quantifying the number of positive cells i.e. those that express GFP either in the whole organism or a part thereof or, in the instance of a mechanically applied inflammatory stimulus having been applied to the organism at the site of injury. In the instance where in vivo exposure to a candidate inflammatory response modulator increases the number of viable granulocytes as compared to the number when not exposed or as a control, the candidate inflammatory response modulator can be identified as having an inflammatory effect. In contrast if the number of viable granulocytes decreases when exposed to a candidate inflammatory response modulator then it can be identified as having an anti-inflammatory response and may be implicated as having a pro-apoptotic effect.

During granulocyte apoptosis, we have demonstrated a loss of GFP fluorescence specifically within neutrophils, and this loss of fluorescence provides a method for the detection of granulocyte apoptosis.

Preferably, the GFP may be modified so as to target the nucleus of the granulocyte, preferably the modification comprises fusing GFP to a nuclear localisation sequence (NLS). This embodiment is particularly useful when assessing nuclear morphology of the granulocyte which undergoes characteristic and easily recognisable changes during apoptosis.

Assessment of viable granulocytes by GFP expression is a particularly preferred embodiment of the present invention as it advantageously provides a simple and rapid quantitative and/or qualitative value.

In a further specific embodiment of the methods of the present invention, the assessment of the amount of viable granulocytes can be achieved by staining, preferably with a granulocyte specific enzyme stain, such as myeloperoxidase. In this embodiment the number of cells staining positively for the presence of myeloperoxidase may be calculated both in vivo and in post-mortem samples of the organism.

In a yet further embodiment of the methods of the present invention, the in vivo assessment of viable granulocytes can be achieved by using transgenically modified aquatic vertebrate organisms that express an apoptosis specific reporter. For example the aquatic vertebrate organism may be transgenically modified so as to express in vivo a granulocyte organelle targeted reporter, for the apoptosis specific reporter for example GFP which may be operably linked to for example a nuclear localisation sequence to allow assessment of viable granulocytes. Similarly, a fusion protein comprising cytochrome C fused to GFP or another reporter will remain constrained within the mitochondria in viable granulocytes and be released into the cytoplasm as apoptosis ensues thereby allowing easy recognition of apoptotic granulocytes. Alternatively, preferably apoptosis related protease activity reporters may be used so that the organisms express a construct which contains an apoptosis related protease sensitive cleavage site and is engineered in such a way that activation of apoptosis related proteases cause a detectable change in the reporter. FRET methodology can be employed for assessment of the amount of viable granulocytes as herein after described in more detail. Preferably, an organelle targeting sequence linked to reporters by a protease sensitive linker sequence can be used to allow for identification of protease activity. Suitable examples of apoptosis related proteases are caspases, cathepsins and calpains.

In a further aspect of the invention there is provided a screening method for the identification of an agent that modulates an inflammatory response comprising:
i) assessing the amount of viable granulocytes in the presence or absence of an applied inflammatory stimulus, in an aquatic vertebrate organism that by virtue of genetic modification or transgenesis possesses a delayed resolution of inflammation phenotype and expresses an apoptotic specific reporter protein or product that can be detected in vivo,
ii) exposing the said genetically modified aquatic vertebrate organism to a candidate modulator agent and assessing the amount of viable granulocytes; and
iii) comparing the amount of viable granulocytes obtained from step i) against those obtained from step ii), whereby a difference in values obtained in step i) and step ii) indicates whether the agent inhibits or enhances the inflammatory response.

It will be appreciated that this particular aspect of the invention is particularly suited to screening genetically abnormal organisms, that are characterised phenotypically by having a delayed resolution of inflammation and the method may be used to screen for compound that correct or compensate for the genetic abnormality.

Preferably, the genetic manipulation is sufficient to achieve a delayed resolution of inflammation phenotype, such manipulations can include, for example and without limitation introduction of a transgene, mutation and gene knock-out. It will be understood that other methods of genetic manipulation may also be used to create an aquatic vertebrate organism that has the desired atypical phenotype.

Preferably, this aspect of the invention includes any one or more features ascribed to other aspects of the invention.

One embodiment of the present invention relates to an assay that utilises fluorescence detection in cells, typically granulocytes, to screen for pro-apoptotic agents. In particular the present disclosure makes use of fluorescence resonance energy transfer (FRET). FRET is a process in which energy is transferred in a non-radiative manner from an excited donor fluorescent reagent to an acceptor fluorescent reagent by means of intermolecular long-range dipole-dipole coupling. FRET typically occurs over distances of about 10 A to 100 A and requires that the emission spectrum of the donor reagent and the absorbance spectrum of the acceptor reagent overlap adequately and that the quantum yield of the donor and the absorption coefficient of the acceptor be sufficiently high. In addition, the transition dipoles of the donor and acceptor fluorescent reagents must be properly oriented relative to one another. For reviews of FRET see Clegg, 1995, Current Opinions in Biotechnology 6: 103-110; Wu & Brand, 1994, Anal. Biochem. 218: 1-13.

According to a further aspect of the invention there is provided a screening method for the identification of an agent that modulates the inflammatory response, the method comprising
i) providing a granulocyte comprising a polypeptide wherein the polypeptide comprises a fluorophore and exposing the granulocyte to a source of light sufficient to excite the fluorophore;
ii) measuring the amount of fluorescence in the granulocyte in the absence of an agent to be tested;
iii) exposing the granulocyte to an agent to be tested and exposing the granulocyte to a source of light sufficient to excite the fluorophore; and
iv) measuring the amount of fluorescence in the cells in the presence of an agent to be tested, and comparing the amount of fluorescence measured in step (iv) to the amount of fluorescence measured in step (ii).

According to a further aspect of the invention there is provided a screening method for the identification of a pro-apoptotic agent the method comprising
i) providing a granulocyte comprising a polypeptide wherein the polypeptide comprises a fluorophore and exposing the granulocyte to a source of light sufficient to excite the fluorophore;
ii) measuring the amount of fluorescence in the granulocyte in the absence of an agent to be tested;
iii) exposing the granulocyte to an agent to be tested and exposing the granulocyte to a source of light sufficient to excite the fluorophore; and

iv) measuring the amount of fluorescence in the cells in the presence of an agent to be tested, wherein if the amount of fluorescence measured in step (iv) is less than the amount of fluorescence measured in step (ii) then the agent is pro-apoptotic.

In a further aspect of the invention provides a screening method for the identification of a pro-apoptotic agent the method comprising
i) forming a preparation comprising a granulocyte wherein the granulocyte includes a nucleic acid molecule comprising
   a) a nucleic acid sequence encoding a fluorophore; and
   b) a nucleic acid sequence comprising all, or the regulatory part thereof, of the promoter sequence for a gene encoding a protein expressed in granulocyte operably linked to the nucleic acid in a) and exposing the preparation to a source of light sufficient to excite the fluorophore;
ii) measuring fluorescence in the granulocyte in the absence of an agent to be tested;
iii) exposing the granulocyte to an agent to be tested and exposing the preparation to a source of light sufficient to excite the fluorophore; and
iv) measuring fluorescence in the cells in the presence of an agent to be tested.

Preferably the amount of fluorescence is measured in steps (ii) and (iv) wherein if the amount of fluorescence measured in step (iv) is less than the amount of fluorescence measured in step (ii) then the agent is pro-apoptotic.

In a preferred method step (i) comprises
i) forming a preparation comprising a granulocyte wherein the granulocyte includes a nucleic acid molecule comprising
   a) a nucleic acid sequence encoding a fluorophore translationally fused to a nuclear localisation sequence motif; and
   b) a nucleic acid sequence comprising all, or the regulatory part thereof, of the promoter sequence for a gene encoding a protein expressed in granulocyte operably linked to the nucleic acid in a);

In a further aspect the invention provides a screening method for the identification of agents that induce granulocyte apoptosis the method comprising the steps of
i) providing a granulocyte comprising a nucleic acid molecule comprising
   a) a nucleic acid sequence encoding a fusion protein wherein the fusion protein comprises a fluorophore and a sub-cellular localisation sequence linked by a peptide comprising a protease cleavage site; and
   b) a nucleic acid sequence comprising all, or the regulatory part thereof, of the promoter sequence for a gene encoding a protein expressed in granulocytes operably linked to the nucleic acid in (a) and exposing the granulocyte to a source of light sufficient to excite the fluorophore;
ii) assessing the amount and distribution of fluorescence in the granulocyte in the absence of an agent to be tested;
iii) exposing the granulocyte to an agent to be tested and exposing the granulocyte to a source of light sufficient to excite the fluorophore;
iv) assessing the amount and distribution of fluorescence in the granulocyte in the presence of an agent to be tested.

Fluorescence is monitored in the sub-cellular organelle in question in the absence and presence of the agent to be tested. If the localisation, or degree of fluorescence, in the sub-cellular organelle in question changes in the presence of the agent to be tested, compared to in the absence of the agent to be tested, then the agent may be a pro-apoptotic agent. Typically loss of localisation of fluorescence in the sub-cellular organelle in question in the presence of the agent to be tested indicates that the agent is a pro-apoptotic agent.

Preferably the sub-cellular localisation sequence is a nuclear localisation sequence, and the sub-cellular organelle is therefore the nucleus.

In a further aspect the invention provides a screening method for the identification of agents that induce granulocyte apoptosis the method comprising the steps of
i) providing a granulocyte comprising a nucleic acid molecule comprising
   a) a nucleic acid sequence encoding a fusion protein wherein the fusion protein comprises a fluorophore engineered to include a peptide comprising a protease cleavage site; and
   b) a nucleic acid sequence comprising all, or the regulatory part thereof, of the promoter sequence for a gene encoding a protein expressed in granulocytes operably linked to the nucleic acid in (a) and (b) and exposing the granulocyte to a source of light sufficient to excite the fluorophore;
ii) measuring the amount of fluorescence in the granulocyte in the absence of an agent to be tested;
iii) exposing the granulocyte to an agent to be tested and exposing the granulocyte to a source of light sufficient to excite the fluorophore;
iv) measuring the amount of fluorescence in the granulocyte in the presence of an agent to be tested.

Fluorescence is monitored in the granulocyte in the absence and presence of the agent to be tested. If the amount of fluorescence measured in the granulocyte in the absence of the agent to be tested is greater than the amount of fluorescence measured in the granulocyte in the presence of the agent to be tested then the agent is a pro-apoptotic agent.

In a further aspect the invention provides a screening method for the identification of agents that induce granulocyte apoptosis the method comprising the steps of
i) providing a granulocyte comprising a vector wherein the vector comprises
   a) a nucleic acid molecule comprising a nucleic acid sequence encoding a fusion protein wherein the fusion protein comprises a fluorophore engineered to include a peptide comprising a protease cleavage site; and
   b) a nucleic acid molecule comprising a nucleic acid sequence encoding a fusion protein wherein the fusion protein comprises a fluorophore;
   c) a nucleic acid sequence comprising all, or the regulatory part thereof, of the promoter sequence for a gene encoding a protein expressed in granulocytes operably linked to the nucleic acid in (a) and (b);
ii) measuring the amount of fluorescence in the granulocyte in the absence of an agent to be tested;
iii) exposing the granulocyte to an agent to be tested;
iv) measuring the amount of fluorescence in the granulocyte in the presence of an agent to be tested.

Preferably the fluorophores in (a) and (b) are different. Fluorescence is monitored in the granulocyte in the absence and presence of the agent to be tested. If the amount of fluorescence emitted by the fluorophore in (ii) is greater than the amount of fluorescence emitted by the fluorophore in (iv) in the presence of the agent to be tested, then the agent is a pro-apoptotic agent.

In a further aspect of the invention there is provided a screening method for the identification of a pro-apoptotic agent the method comprising
i) providing a granulocyte comprising a fusion protein wherein the fusion protein comprises a donor fluorophore and an acceptor fluorophore linked by a peptide comprising a protease cleavage site and exposing the granulocyte to a source of light sufficient to excite the donor fluorophore;
ii) measuring the amount of fluorescence resonance energy transfer (FRET) in the granulocyte in the absence of an agent to be tested;
iii) exposing the granulocyte to an agent to be tested and exposing the granulocyte to a source of light sufficient to excite the donor fluorophore;
iv) measuring the amount of FRET in the granulocyte in the presence of an agent to be tested, wherein if the amount of FRET measured in step (ii) is greater than the amount of FRET measured in step (iv) then the agent is a pro-apoptotic agent.

As used herein a “donor fluorophore” refers to a fluorescent reagent that absorbs and emits energy (fluorescence) at a first wavelength. Energy from the donor fluorophore is transferred to an “acceptor fluorophore” absorbs fluorescence emitted by the donor fluorophore and emits fluorescence at a second wavelength, wherein the first and second wavelengths are different.

In a preferred method of the invention said screening method includes the steps of: collating the activity data in (ii) and (iv) above; converting the collated data into a data analysable form; and optionally providing an output for the analysed data.

A number of methods are known which image fluorescent cells and extract information concerning the spatial and temporal changes occurring in cells expressing fluorescent proteins, (see Taylor et al Am. Scientist 80: 322-335, 1992), which is incorporated by reference. Moreover, U.S. Pat. No. 5,989,835 and U.S. Ser. No. 09/031,271, both of which are incorporated by reference, disclose optical systems for determining the distribution or activity of fluorescent reporter molecules in cells for screening large numbers of agents for biological activity. The systems disclosed in the above patents also describe a computerised method for processing, storing and displaying the data generated.

The screening of large numbers of agents requires preparing arrays of cells for the handling of cells and the administration of agents. Assay devices, for example, include standard multiwell microtitre plates with formats such as 6, 12, 48, 96 and 384 wells which are typically used for compatibility with automated loading and robotic handling systems. Typically, high throughput screens use homogeneous mixtures of agents with an indicator compound which is either converted or modified resulting in the production of a signal. The signal is measured by suitable means (for example detection of fluorescence emission, optical density, or radioactivity) followed by integration of the signals from each well containing the cells, agent and indicator compound.

Thus in a further aspect the invention provides a method of high throughput screening to identify a pro-apoptotic agent the method comprising: repeating at least 5,000 times in a 24 hour period the steps of:
i) providing a granulocyte comprising a fusion protein wherein the fusion protein comprises a donor fluorophore and an acceptor fluorophore linked by a peptide comprising a protease cleavage site and exposing the granulocyte to a source of light sufficient to excite the donor fluorophore;
ii) measuring the amount of FRET in the granulocyte in the absence of an agent to be tested;
iii) exposing the granulocyte to an agent to be tested and exposing the granulocyte to a source of light sufficient to excite the donor fluorophore;
iv) measuring the amount of FRET in the granulocyte in the presence of an agent to be tested, wherein if the amount of FRET measured in step (ii) is greater than the amount of FRET measured in step (iv) then the agent is a pro-apoptotic agent;
   wherein the treated granulocytes are exposed to at least 5,000 different agents to be tested in the 24 hour period.

Examples of fluorophores, including donor-acceptor fluorophores, include green fluorescent proteins and derivatives thereof, Alexa488-Alexa555; Alexa488-Cy3; FITC-TRITC; DiSBAC4(3)-CC2-DMPE.

Preferably the fluorophore, such as donor-acceptor fluorophores (FRET pairs), is/are green fluorescent proteins (GFPs) or derivatives thereof. Fluorescence emission spectrum shifted derivatives of GFP may include blue fluorescent protein (BFP) and yellow fluorescent protein (YFP). Other derivatives include enhanced cyan yellow protein (ECYP), EYFP, EGFP. Other examples of FRET pairs include BFP-GFP; CFP-dsRED; BFP-GFP; Cy3-Cy5; CFP-YFP. Preferably the donor GFP is CFP and the acceptor GFP is YFP. YFP has an excitation/emission wavelength of 500/535 nm. CFP has an excitation/emission wavelength of 436/470 nm.

A green fluorescent protein (GFP) is a fluorescent protein in which any 150 contiguous amino acids have an amino acid sequence identity of at least 85% to a contiguous stretch of the amino acid sequence of wild-type GFP known in the art.

Preferably the fusion protein is a polypeptide comprising a sequence of amino acids in which two green fluorescent proteins (GFPs) are joined by a linker that consists of a short stretch of amino acids where the linker comprises at least one protease cleavage site. One GFP is a donor GFP and the other GFP is an acceptor GFP. The two GFPs are different GFPs such that fluorescence resonance energy transfer (FRET) can occur from the donor GFP to the acceptor GFP when the donor GFP is excited and when the linker is intact but cleavage of the linker separates the donor and acceptor GFPs and FRET is abolished or greatly diminished.

Either the donor or the acceptor GFP may be at the amino or the carboxy terminal portion of the fusion protein. Also, additional peptide sequences may be present at the amino or carboxy terminal ends of the fusion proteins.

The protease capable of cleaving the peptide at the protease cleavage site may be a pro-apoptotic protein, for example a calpain, cathepsin, caspase (e.g. caspases-1, -3, and -8). In a preferred method of the invention the protease cleavage site is a caspase cleavage site, for example the caspase recognition sequence DEVD.

To measure FRET a technique called radiometric analysis is used and involves determining the ratio of the fluorescence emitted by an acceptor fluorophore (GFP) divided by the fluorescence emitted by a donor fluorophore (GFP) after excitation of the donor fluorophore (GFP).

In a further aspect the invention provides a screening method for the identification of agents that induce granulocyte apoptosis the method comprising the steps of
i) providing a granulocyte comprising a nucleic acid molecule comprising
   a) a nucleic acid sequence encoding a fusion protein wherein the fusion protein comprises a donor fluorophore and an acceptor fluorophore linked by a peptide comprising a protease cleavage site and exposing the granulocyte to a source of light sufficient to excite the fluorophore; and
   b) a nucleic acid sequence comprising all, or the regulatory part thereof, of the promoter sequence for a gene encoding a protein expressed in granulocytes operably linked to the nucleic acid in (a);
ii) measuring the amount of FRET in the granulocyte in the absence of an agent to be tested;
iii) exposing the granulocyte to an agent to be tested and exposing the granulocyte to a source of light sufficient to excite the fluorophore;
iv) measuring the amount of FRET in the granulocyte in the presence of an agent to be tested, wherein if the amount of FRET measured in step (ii) is greater than the amount of FRET measured in step (iv) then the agent is a pro-apoptotic agent.

In a preferred method the promoter is for a neutrophil specific gene. Preferably the neutrophil specific gene is a zebrafish gene. Preferably the promoter is the neutrophil specific myeloperoxidase promoter. The MPO gene has been cloned; a BAC containing the MPO gene promoter is BAC zC 91B8 and the sequence is publicly available at http://www.sanger.ac.uk/Projects/D_rerio/wgs.shtml

In a further aspect the invention provides a screening method for the identification of agents that induce granulocyte apoptosis the method comprising the steps of

i) providing a preparation comprising a first granulocyte in the absence of an agent to be tested wherein said granulocyte comprises
a) a nucleic acid sequence encoding a fusion protein wherein the fusion protein comprises a donor fluorophore and an acceptor fluorophore linked by a peptide comprising a protease cleavage site; and
b) a nucleic acid sequence comprising all, or the regulatory part thereof, of the promoter sequence for a gene encoding a protein expressed in granulocytes operably linked to the nucleic acid in (a) and exposing the preparation to a source of light sufficient to excite the donor fluorophore; and
   ii) providing a preparation comprising a second granulocyte in the presence of an agent to be tested wherein said granulocyte comprises
a) a nucleic acid sequence encoding a fusion protein wherein the fusion protein comprises a donor fluorophore and an acceptor fluorophore linked by a peptide comprising a protease cleavage site; and
b) a nucleic acid sequence comprising all, or the regulatory part thereof, of the promoter sequence for a gene encoding a protein expressed in granulocytes operably linked to the nucleic acid in (a) and exposing the granulocyte to a source of light sufficient to excite the donor fluorophore;
   ii) comparing the amount of FRET measured in the first granulocyte preparation with the amount of FRET measured in the second granulocyte preparation wherein if the amount of FRET measured in the first granulocyte preparation is greater than the amount of FRET measured in second granulocyte preparation then the agent is a pro-apoptotic agent.

A further aspect of the invention provides a transcription cassette comprising a nucleic acid molecule comprising
a) a nucleic acid sequence encoding a fluorophore; and
b) a nucleic acid sequence comprising all, or the regulatory part thereof, of the promoter sequence for a gene encoding a protein expressed in granulocyte operably linked to the nucleic acid in a).

Preferably the transcription cassette of the invention comprises
a) a nucleic acid sequence encoding a fluorophore;
b) a nuclear localisation sequence operably linked to the nucleic acid sequence in a); and
c) a nucleic acid sequence comprising all, or the regulatory part thereof, of the promoter sequence for a gene encoding a protein expressed in granulocyte operably linked to the nucleic acid in a).

A further aspect of the invention comprises a transcription cassette comprising a nucleic acid molecule comprising
a) a nucleic acid sequence encoding a fusion protein wherein the fusion protein comprises fluorophore and a sub-cellular localisation sequence linked by a peptide comprising a protease cleavage site; and
b) a nucleic acid sequence comprising all, or the regulatory part thereof, of the promoter sequence for a gene encoding a protein expressed in granulocytes operably linked to the nucleic acid in (a).

A further aspect of the invention comprises a transcription cassette comprising a nucleic acid molecule comprising
a) a nucleic acid sequence encoding a fusion protein wherein the fusion protein comprises a fluorophore engineered to include a peptide comprising a protease cleavage site; and
b) a nucleic acid sequence comprising all, or the regulatory part thereof, of the promoter sequence for a gene encoding a protein expressed in granulocytes operably linked to the nucleic acid in (a) and (b).

A further aspect of the invention provides a transcription cassette comprising a nucleic acid molecule comprising
i) a nucleic acid sequence encoding a fusion protein wherein the fusion protein comprises a donor fluorophore and an acceptor fluorophore linked by a peptide comprising a protease cleavage site; and
ii) a nucleic acid sequence comprising all, or the regulatory part thereof, of the promoter sequence for a gene encoding a protein expressed in granulocytes operably linked to the nucleic acid in (i).

The transcription cassettes described herein may be part of a vector adapted for recombinant expression of said nucleic acid molecules. Thus, in a further aspect, the invention provides a vector comprising a transcription cassette of the invention.

In a further aspect there is a provided a cell comprising one or more vectors according to the invention. The vector may be stably or transiently transfected into the cell. Preferably the cell is a granulocyte.

A further aspect of the invention provides a vector comprising a nucleic acid molecule comprising
a) a nucleic acid molecule comprising a nucleic acid sequence encoding a fusion protein wherein the fusion protein comprises a fluorophore engineered to include a peptide comprising a protease cleavage site;
b) a nucleic acid molecule comprising a nucleic acid sequence encoding a fusion protein wherein the fusion protein comprises a fluorophore; and
c) a nucleic acid sequence comprising all, or the regulatory part thereof, of the promoter sequence for a gene encoding a protein expressed in granulocytes operably linked to the nucleic acid in (a) and (b);

A further aspect of the invention provides a granulocyte transfected with a nucleic acid molecule wherein the nucleic acid molecule comprises
i) a nucleic acid sequence encoding a fusion protein wherein the fusion protein comprises a donor fluorophore and an acceptor fluorophore linked by a peptide comprising a protease cleavage site; and
ii) a nucleic acid sequence comprising all, or the regulatory part thereof, of the promoter sequence for a gene encoding a protein expressed in granulocytes operably linked to the nucleic acid in (i).

Preferably the nucleic acid is DNA.

A yet further aspect of the invention provides a non-human organism comprising a granulocyte transfected with a vector according to the invention. Preferably the vector is adapted for expression of a nucleic acid molecule wherein the nucleic acid molecule comprises
i) a nucleic acid sequence encoding a fusion protein wherein the fusion protein comprises a donor fluorophore and an acceptor fluorophore linked by a peptide comprising a protease cleavage site; and
ii) a nucleic acid sequence comprising all, or the regulatory part thereof, of the promoter sequence for a gene encoding a protein expressed in granulocytes operably linked to the nucleic acid in (i).

Preferably the organism is an aquatic vertebrate organism such as the zebrafish.

The invention further provides a transgenic zebrafish which has been genetically manipulated to express a transcription cassette, or vector, according to the invention. Preferably the vector is adapted for expression of a nucleic acid molecule wherein the nucleic acid molecule comprises
i) a nucleic acid sequence encoding a fusion protein wherein the fusion protein comprises a donor fluorophore and an acceptor fluorophore linked by a peptide comprising a protease cleavage site; and
ii) a nucleic acid sequence comprising all, or the regulatory part thereof, of the promoter sequence for a gene encoding a protein expressed in granulocytes operably linked to the nucleic acid in (i).

The invention further provides a method of screening method for the identification of a pro-apoptotic agent the method comprising measuring the amount of FRET in a transgenic aquatic vertebrate organism such as a zebrafish according to the invention in the absence and presence of an agent to be tested. If the amount of FRET measured in the zebrafish in the absence of the agent to be tested is greater than the amount of FRET measured in the zebrafish in the presence of the agent to be tested, then said agent is pro-apoptotic.

A yet further aspect of the invention provides a method of screening method for the identification of a pro-apoptotic agent the method comprising the steps of
i) providing a transgenic zebrafish according to the invention;
ii) measuring the amount of fluorescence, for example FRET, in the zebrafish in the absence of an agent to be tested;
iii) administering an agent to be tested to said zebrafish;
iv) measuring the amount of fluorescence, for example FRET, in the zebrafish in the presence of an agent to be tested.

If the amount of FRET measured in step (ii) is greater than the amount of FRET measured in step (iv) then the agent is a pro-apoptotic agent.

Because of the small size and permeability of larvae, zebrafish are amenable to use in screens for biological effects of small molecules. Candidates may be administered by pipetting into 96 well plates in which larvae are arrayed.

In a further aspect the invention provides the use of a nucleic acid molecule in the identification of agents that induce granulocyte, for example neutrophil, apoptosis wherein the nucleic acid molecule comprises
a) a nucleic acid sequence encoding a fusion protein wherein the fusion protein comprises a fluorophore optionally linked to, or engineered to include, a peptide comprising a protease cleavage site; and
b) a nucleic acid sequence comprising all, or the regulatory part thereof, of the promoter sequence for a gene encoding a protein expressed in granulocytes operably linked to the nucleic acid in (a).

In one embodiment of the method of the invention said agent is an antibody, preferably a monoclonal antibody or modified antibody, or at least the effective binding part thereof.

Antibodies, also known as immunoglobulins, are protein molecules which usually have specificity for foreign molecules (antigens). Immunoglobulins (Ig) are a class of structurally related proteins consisting of two pairs of polypeptide chains, one pair of light (L) (low molecular weight) chain (ic or %), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant.

The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region.

The H chains of Ig molecules are of several classes, α, μ, σ, α, and γ (of which there are several sub-classes). An assembled Ig molecule consisting of one or more units of two identical H and L chains, derives its name from the H chain that it possesses. Thus, there are five Ig isotypes: IgA, IgM, IgD, IgE and IgG (with four sub-classes based on the differences in the ‘constant’ regions of the H chains, i.e., IgG1, IgG2, IgG3 and IgG4). Further detail regarding antibody structure and their various functions can be found in, Using Antibodies: A laboratory manual, Cold Spring Harbour Laboratory Press.

In a preferred method of the invention said fragment is a Fab fragment.

In a further preferred method of the invention said antibody is selected from the group consisting of: F(ab′)₂, Fab, Fv and Fd fragments; and antibodies comprising CDR3 regions.

Preferably said fragments are single chain antibody variable regions (scFV's) or domain antibodies. If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

A modified antibody, or variant antibody and reference antibody, may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants which show enhanced biological activity.

Preferably said antibody is a humanised or chimeric antibody.

A chimeric antibody is produced by recombinant methods to contain the variable region of an antibody with an invariant or constant region of a human antibody.

A humanised antibody is produced by recombinant methods to combine the complementarity determining regions (CDRs) of an antibody with both the constant (C) regions and the framework regions from the variable (V) regions of a human antibody.

Chimeric antibodies are recombinant antibodies in which all of the V-regions of a mouse or rat antibody are combined with human antibody C-regions. Humanised antibodies are recombinant hybrid antibodies which fuse the complimentarity determining regions from a rodent antibody V-region with the framework regions from the human antibody V-regions. The C-regions from the human antibody are also used. The complimentarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (i.e. foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In an alternative preferred method of the invention said agent is a polypeptide or a peptide. Preferably said polypeptide or peptide is modified.

In a preferred method of the invention said peptide is at least 6 amino acid residues in length. Preferably the length of said peptide/polypeptide is selected from the group consisting of: at least 7 amino acid residues; 8, 9, 10, 11, 12, 13, 14, 15, 16, 17, 18, 19, or 20 amino acid residues in length. Alternatively the length of said peptide/polypeptide is at least 20 amino acid residues; 30; 40; 50; 60; 70; 80; 90; or 100 amino acid residues in length.

It will be apparent to one skilled in the art that modification to the amino acid sequence of peptide agents could enhance the binding and/or stability of the peptide with respect to its target sequence. In addition, modification of the peptide may also increase the in vivo stability of the peptide thereby reducing the effective amount of peptide necessary to inhibit the activity of a target polypeptide. This would advantageously reduce undesirable side effects which may result in vivo. Alternatively or preferably, said modification includes the use of modified amino acids in the production of recombinant or synthetic forms of peptides. It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (e.g. F, Br, I), hydroxy or C₁-C₄ alkoxy. Modifications also include, by example and not by way of limitation, acetylation and amidation.

In a preferred embodiment of the invention said peptide sequence is acetylated. Preferably said acetylation is to the amino terminus of said peptide.

In a further preferred embodiment of the invention said peptide sequence is amidated. Preferably said amidation is to the carboxyl-terminus of said peptide.

It will also be apparent to one skilled in the art that peptides could be modified by cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

In a further preferred method of the invention said agent is nucleic acid molecule. Preferably said nucleic acid molecule is an aptamer or a modified aptamer.

Nucleic acids have both linear sequence structure and a three dimensional structure which in part is determined by the linear sequence and also the environment in which these molecules are located. Conventional therapeutic molecules are small molecules, for example, peptides, polypeptides, or antibodies, which bind target molecules to produce an agonistic or antagonistic effect. It has become apparent that nucleic acid molecules also have potential with respect to providing agents with the requisite binding properties which may have therapeutic utility. These nucleic acid molecules are typically referred to as aptamers. Aptamers are small, usually stabilised, nucleic acid molecules which comprise a binding domain for a target molecule. A screening method to identify aptamers is described in U.S. Pat. No. 5,270,163, which is incorporated by reference. Aptamers are typically oligonucleotides which may be single stranded oligodeoxynucleotides, oligoribonucleotides, or modified oligodeoxynucleotide or oligoribonucleotides.

The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include by example and not by way of limitation; alkylated purines and/or pyrimidines; acylated purines and/or pyrimidines; or other heterocycles. These classes of pyrimidines and purines are known in the art and include, pseudoisocytosine; N4,N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine.

The aptamers of the invention are synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—. The binding of aptamers to a target polypeptide is readily testable.

In an alternative preferred method of the invention said agent is a small molecule, for example, a molecule isolated and characterised by combinatorial chemistry.

According to a further aspect of the invention there is provided an antibody identified by the method according to the invention for use as a pharmaceutical, especially for the modulation of the inflammatory response.

According to a further aspect of the invention there is provided a polypeptide or peptide identified by the method according to the invention for use as a pharmaceutical, especially for the modulation of the inflammatory response.

According to a further aspect of the invention there is provided a nucleic acid molecule identified by the method according to the invention for use as a pharmaceutical, especially for the modulation of the inflammatory response.

In a preferred embodiment of the invention said nucleic acid molecule is an aptamer.

In a further aspect the invention provides an agent identified by the screening method of the invention. Preferably, the agent is one which influences, directly or indirectly, the outcome of the inflammatory response.

In a further aspect there is provided an agent identified by the screening method of the invention for use as a pharmaceutical, especially for the modulation of the inflammatory response.

The agent may be useful in the treatment of prevention of neutrophil associated inflammatory disorders such as asthma, ARDS, COPD, Interstitial Lung disease, Rheumatoid arthritis and other inflammatory arthritides, reperfusion injury following coronary or cerebral artery occlusion (heart attack and stroke).

Preferred features of each and every aspect of the invention are as for each of the other aspects mutatis mutandis.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

FIG. 1 shows a map of BAC C 91B8 which has been modified to include in-frame GFP;

FIG. 2 shows a map of BAC C 91B8 which has been modified to include in-frame GFPnuc (GFP fused to a Nuclear localisation sequence;

FIG. 3 shows a map of BAC C 91B8 which has been modified to include in-frame CFP-DEVD-YFP;

FIG. 4 shows a view of the head of a day 4 transgenic zebrafish larva clearly shows the distribution of neutrophils in the region.

FIG. 5 shows neutrophil distribution in a section of zebrafish tail fin following injury. The image proceeds in 15 minute intervals from left to right and continues on the second row.

FIG. 6 shows neutrophil distribution in a section of zebrafish tail fin following injury. The images, approximately 2 minutes apart, are shown from left to right, and continued on the second row.

FIG. 7 shows bar charts of the number of neutrophils up to 24 hours post an applied inflammatory stimulus as assessed by manual counting (FIG. 7a) and by automated image analysis (FIG. 7b).

FIG. 8 shows results of manipulation of the inflammatory response by granulocyte apoptosis in transgenic zebrafish with a caspase inhibitor (FIG. 8a) LPS (FIG. 8b) and pyocyanin (FIG. 8c).

### DETAILED DESCRIPTION

### Materials and Methods

AB strain zebrafish from existing aquarium stocks were maintained according to standard protocols. A BAC containing approx 130 kB of 5′ promoter sequence to the MPO gene was identified (zC91B8). This BAC was modified to contain an in-frame GFP sequence beginning at the translation start site of the MPO gene by recombinase activity as previously described (Lee, E. C. et al (2001) Genomics 73, 56-65). EL250 cells required for the recombination step were a kind gift of Dr. Neal Copeland, National Cancer Institute, Frederick, Mass., USA. Insertion of the GFP sequence was confirmed by co-insertion of a kanamycin resistance gene, which in turn was flanked by FRT sites leading to its easy removal before introduction into the zebrafish. As part of this process a vector was created consisting of a truncated portion of the Bluescript vector, into which we had blunt cloned a construct consisting of GFP and the FRT-flanked Kanamycin resistance gene, all of which was flanked by MPO homology arms. Using this construct, we were able to add a Nuclear Localising Sequence corresponding to that used in the Clontech vector pEYFPnuc by PCR and self ligation.

Similar modified BACs were generated using GFPnuc and CFP-DEVD-YFP in place of GFP.

The CFP-DEVD-YFP construct was a kind gift of Professor J. Tavare, University of Bristol, and was in turn generated by subcloning of CFP and YFP from the appropriate Clontech Living Colours vector (Tyas, L. et al (2000) EMBO Rep 1, 266-70).

Imaging is performed on a Ni0kon TE-2000S with excitation and emission filter wheels using the IP lab software (Scanalytics, Rockville, Md., USA). Net FRET signal is calculated using the method of Gordon, G. W. et al (1998) Biophys J 74, 2702-13) using the RatioPlus plug in (Scanalytics).

The modified BACs were injected into fertilised AB zebrafish embryos at the 1-2 cell stage, and larvae screened at 36-48 hours for the presence of fluorescence. Fluorescent larvae were grown to maturity and either in- or out-crossed to identify germ cell carriage of the transgene.

Studies are underway characterising these lines, and to use them to identify apoptotic neutrophils.

### Example 1

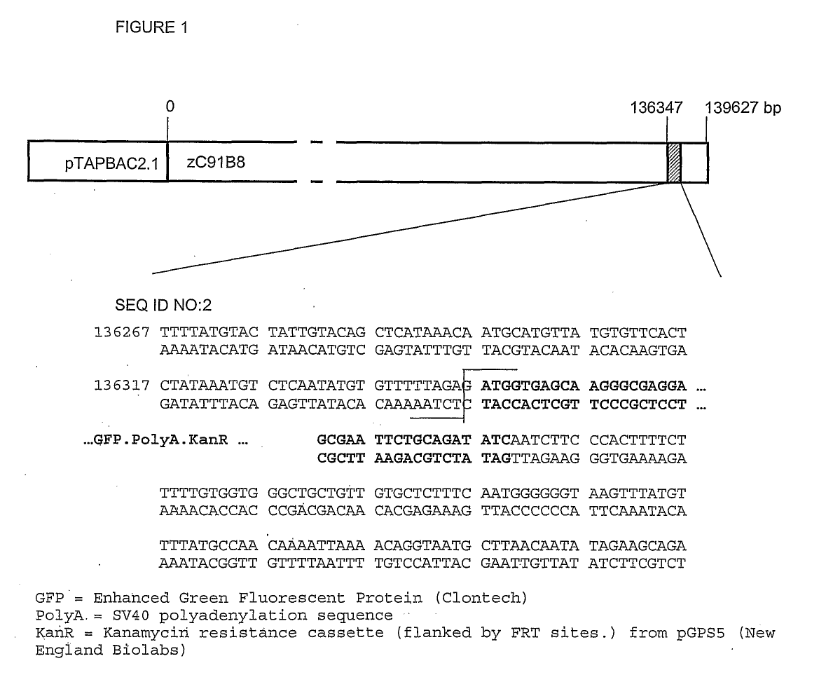

A BAC (zC91B8 in pTAPBAC2.1) was modified by the use of a red recombinase system in EL250 cells (gift of Dr. Neal Copeland, National Cancer Institute, Frederick, Mass.)⁵. This BAC, linearized with PI-Sce1, and was used to generate stable transgenic lines according to published protocols. FIGS. 1-3 show the linearised construct (not to scale) with the distance from the origin of the BAC zC91B8 indicated in base pairs. The shaded region corresponds to the modified sequence, and the sequence is expanded below each Figure. The exact sequence around the ATG is shown, and the ends of the construct shown in bold. The contents of the construct are shown descriptively, rather than by sequence, and expanded below each figure. The Kanamycin resistance cassette is removed by recombinase action prior to generation of zebrafish lines (ref. Lee et al Genomics)

With regard to FIG. 1, there is shown the BAC as described herein before that has been modified to contain enhanced GFP and the SV40 plolyadenylation signal from pEGFPC1 (Clontech), and the Kanamycin resistance cassette (flanked by FRT sites.) from pGPS5 (New England Biolabs).

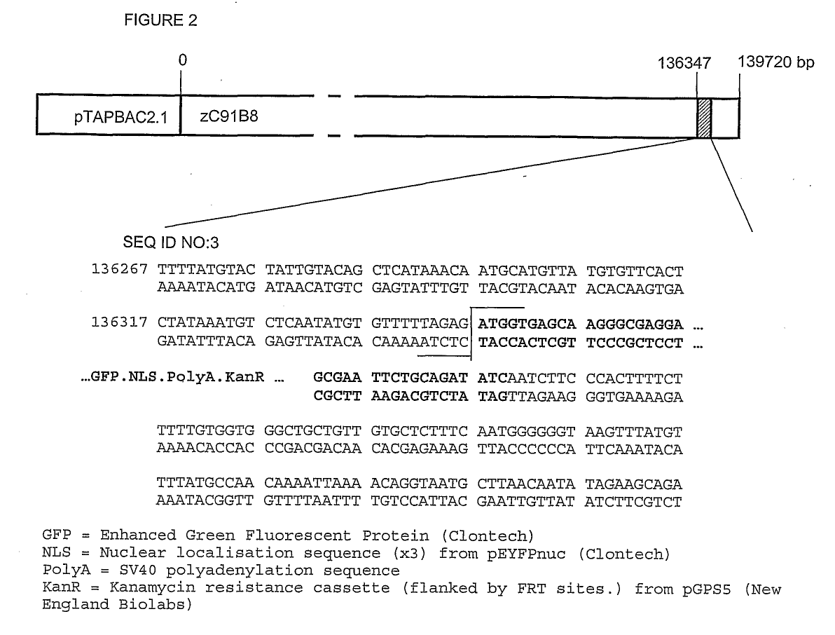

With regard to FIG. 2, there is shown the BAC as described herein before that has been modified to contain enhanced GFP (as above) followed by the nuclear localization sequence (NLS) from pYFPnuc (Clontech), the SV40 plolyadenylation signal (as above), and the Kanamycin resistance cassette (flanked by FRT sites.) from pGPS5 (New England Biolabs).

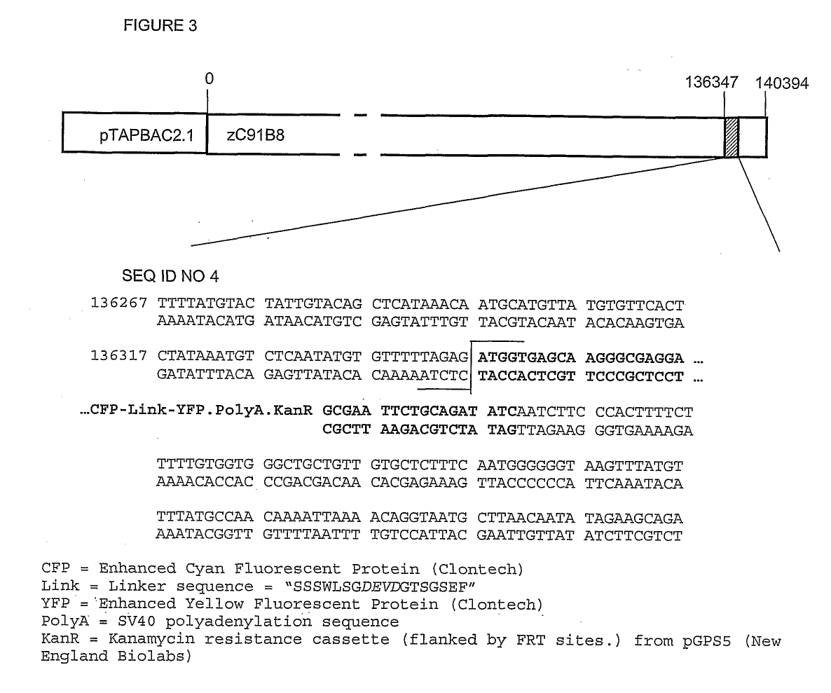

With regard to FIG. 3, there is shown the BAC as described herein before that has been modified to contain enhanced CFP (clontech) followed by a linker sequence “SSSWLSGDEVDGTSGSEF” (SEQ ID NO:1) followed by enhanced YFP (Clontech), the SV40 plolyadenylation signal (as above), and the Kanamycin resistance cassette (flanked by FRT sites.) from pGPS5 (New England Biolabs). The CFP-linker-YFP construct was supplied by Prof. J. Tavare, Bristol and was modified by removal of the N-terminal myc tag before use in this construct (ref. Tyas et al).

### Example 2

We have successfully generated transgenic zebrafish lines expressing GFP under the myeloperoxidase (MPO) promoter, by modification of a BAC as described above.

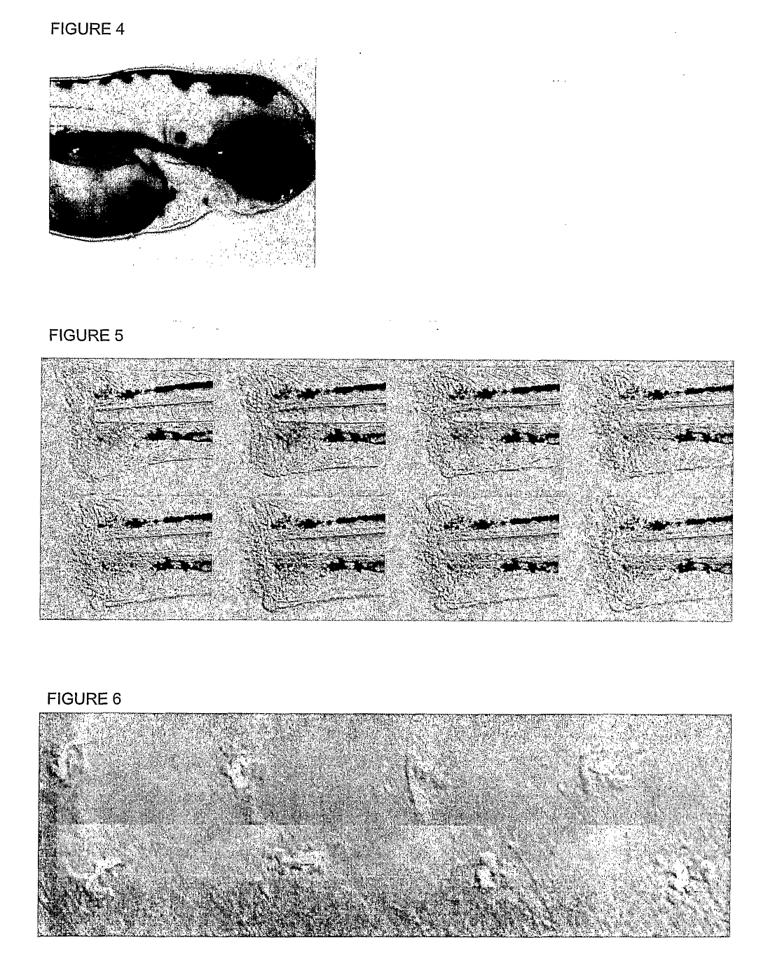

A BAC containing over 100 kb of sequence 5′ to the Zebrafish MPO promoter was identified and modified to contain an in frame GFP sequence (FIG. 3). This was then injected into zebrafish embryos at the 1-cell stage and a transgenic line created. GFP expression recapitulates the expression of myeloperoxidase in cells whose morphology is consistent with neutrophil granulocytes (see FIG. 4). When these fish are injured, neutrophils accumulate at the site of injury over time (FIGS. 5 and 6).

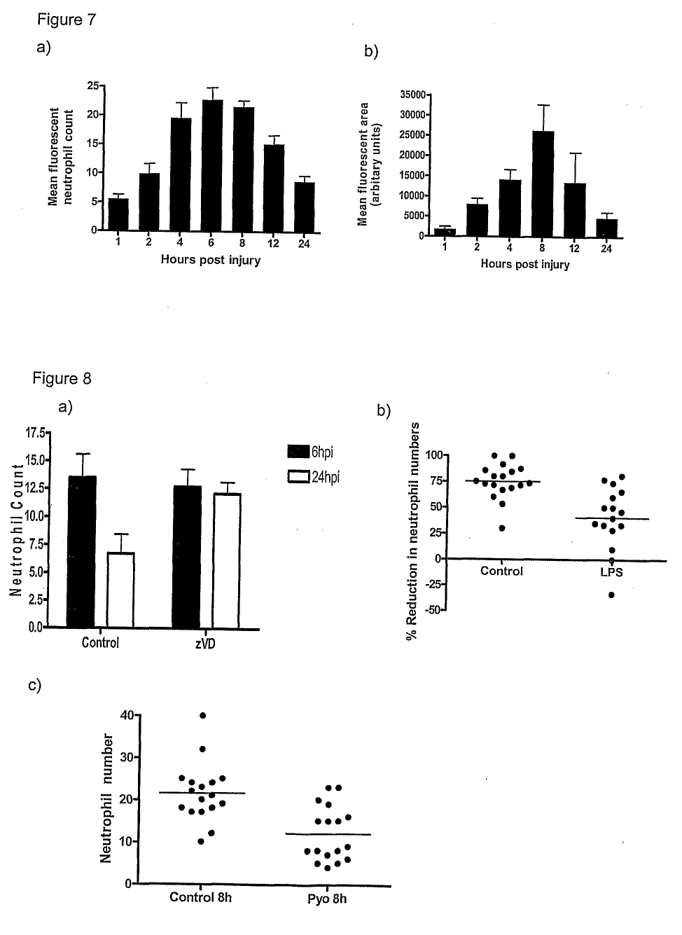

Neutrophil number at the site of injury following tail transection can be assessed either by manual counting (FIG. 7a) or by automated image analysis (FIG. 7b).

Manipulation of the inflammatory response in transgenic zebrafish has been performed exemplifying the role of manipulation of granulocyte apoptosis in regulating the resolution of inflammation. This data is shown in FIG. 8. In FIG. 8a, a caspase inhibitor, zVD.fmk (Calbiochem) delays apoptosis such that there is a failure of the inflammation to resolve, as characterised by a persistence of neutrophil numbers at the inflammatory site between 6 hours and 24 hours, during which time inflammation (as assessed by neutrophil quantification) resolves. P<0.05 for t=24, zVD.fmk treated vs all other groups (1 way ANOVA with Bonferroni post test correction).

In FIG. 8b counts of fluorescent cells in individual larvae were performed for fish in the presence or absence of the bacterial product Lipopolysaccharide (LPS). LPS was added at 6 hours post injury (hpi), and counts made at 6 and 24 hpi. There is a significantly lower % reduction in neutrophil numbers over the time interval 6 to 24 hours in LPS treated fish (p<0.05). In FIG. 8c, Pyocyanin (a bacterial pigment with pro-apoptotic effects on neutrophils) was added at 4 hpi, and counts made at 8 hpi. Absolute cell counts are shown, demonstrating the ability of pyocyanin to reduce neutrophil numbers. p values were calculated using an unpaired 2-tailed t-test.

### Example 3

In addition, we have made constructs expressing two different methods for the in vivo detection of neutrophil apoptosis. The first of these is nuclear targeted GFP (FIG. 2) under the myeloperoxidase promoter. Neutrophil nuclear morphology is characteristic, and undergoes stereotyped changes during apoptosis, thus nuclear targeted GFP gives an instant in vivo readout of apoptosis where it is present. The second technique utilises Fluorescent Resonance Energy Transfer (FRET). FRET technology allows identification of proximity of two transfected fluorescent proteins (YFP and CFP). A FRET construct has been designed that contains YFP and CFP (FIG. 1) joined by a linker sequence sensitive to cleavage by zebrafish caspase-3 (previously described for human caspases: YFP-DEVD-CFP (Tyas, L. et al (2000) EMBO Rep 1, 266-70). Caspase-3 is known to be a major executioner caspase in neutrophils, and is activated during apoptosis (Weinmann, P. et al Blood 93, 3106-3115). The optimal cleavage sequence has been confirmed by comparison of the appropriate region of zebrafish caspase-3 substrates with the comparable sequences in human (eg. PARP, other caspases). In this system, net FRET signal can be calculated, and by comparing YFP fluorescence with the FRET signal, the level of caspase-3 activation in neutrophils can be assessed. When assessing apoptosis, it is considered best practice to use two different and unrelated methods and the ability to assess any positive ‘hits’ using the complementary techniques of caspase activity and nuclear morphology will be an additional advantage. The ability of this system to reliably distinguish apoptotic from non-apoptotic cells is currently being evaluated.

We have generated the expression constructs for both GFPnuc and FRET reporters, and are in the process of generating stable transgenic lines.

This system is suitable for the large scale screening of chemical libraries, either automated or manually. Zebrafish would be arrayed in 100 microlitres of medium in 96 well plates, and suitable concentrations of chemicals added mechanically to each well. After a given time-point (to be determined by preliminary experiment) each well would be examined using a high power fluorescence microscope to identify whether the neutrophils were apoptotic or normal (see FIG. 1 for example of normal distribution of neutrophils). Compounds inducing neutrophil apoptosis will be tested in more rigorous assays for neutrophil apoptosis and toxicity, in both zebrafish and human systems.

1.-62. (canceled)

63. A screening method for the identification of an agent that modulates an inflammatory response comprising:
i) assessing the amount of viable granulocytes in a transgenic aquatic vertebrate organism that expresses a reporter protein or product such that granulocyte apoptosis can be detected in vivo, in the presence or absence of an applied inflammatory stimulus;
ii) exposing the said transgenic aquatic vertebrate organism to a candidate modulator agent and assessing the amount of viable granulocytes; and
iii) comparing the amount of viable granulocytes obtained from step i) against those obtained from step ii), whereby a difference in values obtained in step i) and step ii) indicates whether the agent inhibits or enhances the inflammatory response.

64. A method according to claim 63 wherein steps i) and ii) are performed in reverse order.

65. A method according to claim 63 wherein the applied inflammatory stimulus is selected from the group comprising mechanical damage, stress, hypoxia, disturbances of homeostatic balance, infection, disease and chemical insult by a natural or synthetic agent.

66. A method according to claim 63 wherein assessment of the amount of viable granulocytes is either a qualitative or quantitative assessment.

67. A method according to claim 63 wherein the assessment of viable granulocytes is either in the whole organism or a part of the organism.

68. A method according to claim 63 wherein the granulocyte is selected from the group comprising a mast cell (or mastocyte), basophil, eosinophil, neutrophil or heterophil.

69. A method according to claim 68 wherein the granulocyte is a neutrophil.

70. A method according to claim 63 wherein the aquatic vertebrate organism is a fish or amphibian.

71. A method according to claim 70 wherein the fish is a zebrafish and the amphibian is a xenopus.

72. A method according to claim 63 wherein the screening method is carried out in vivo and/or post mortem.

73. A method according to claim 63 wherein the apoptotic specific reporter protein is a granulocyte specific reporter protein or product that can be detected either directly or indirectly in vivo.

74. A method according to claim 73 wherein expression of the reporter protein or product is under control or is driven by a granulocyte specific promoter.

75. A method according to claim 74 wherein the granulocyte specific promoter is a mycloperoxidase promoter.

76. A method according to claim 73 wherein the reporter protein is a fluorescent protein.

77. A method according to claim 76 wherein the fluorescent protein is selected from the group comprising GFP, eGFP, RCFP, CFP, RFP and YFP.

78. A method according to claim 77 wherein the fluorescent protein is GFP.

79. A method according to claim 76 wherein the fluorescing protein is modified so as to target a granulocyte nucleus.

80. A method according to claim 79 wherein the modification comprises fusing the fluorescent protein encoding sequence to a nuclear localisation sequence (NLS).

81. A method according to claim 63 wherein assessment of the amount of viable granulocytes is achieved by counting or quantifying granulocytes that express a fluorescent protein.

82. A method according to claim 81 wherein the fluorescent protein is GFP.

83. A method according to claim 63 wherein assessment of the amount of viable granulocytes can be achieved by assessment of degree of staining.

84. A method according to claim 83 wherein the stain is a granulocyte specific enzyme stain.

85. A method according to claim 84 wherein the granulocyte specific enzyme stain is myeloperoxidase.

86. A method according to claim 63 wherein the apoptosis specific reporter is GFP and is further linked to an organelle targeting sequence.

87. A method according to claim 63 wherein the aquatic vertebrate organism is a transgenic organism that expresses a construct comprising reporter protein or product having an apoptosis related protease sensitive cleavage site whereby activation of apoptosis related proteases results in a detectable change in activity of the reporter protein or product so that it can be detected in vivo.

88. A method according to claim 87 wherein the apoptosis related protease activity reporter protein is selected from the group consisting of calthains, cathepsins calpains and caspases.

89. A screening method for the identification of an agent that modulates an inflammatory response comprising:
i) assessing the amount of viable granulocytes in the presence or absence of an applied inflammatory stimulus, in a transgenic aquatic vertebrate organism that has been genetically modified so that it possesses a delayed resolution of inflammation phenotype and expresses a reporter protein or product such that granulocyte apoptosis can be detected in vivo,
ii) exposing the said genetically modified aquatic vertebrate organism to a candidate modulator agent and assessing the amount of viable granulocytes; and
iii) comparing the amount of viable granulocytes obtained from step i) against those obtained from step ii), whereby a difference in values obtained in step i) and step ii) indicates whether the agent inhibits or enhances the inflammatory response.

90. A method for the identification of an agent that modulates the inflammatory response, the method comprising
i) providing a granulocyte obtainable from a genetically modified aquatic vertebrate organism, the granulocyte comprising a fusion protein wherein the fusion protein comprises a donor fluorophore and an acceptor fluorophore linked by a peptide comprising a protease cleavage site;
ii) measuring the amount of fluorescence resonance energy transfer (FRET) in the granulocyte in the absence of an agent to be tested;
iii) exposing the granulocyte to an agent to be tested;
iv) measuring the amount of FRET in the granulocyte in the presence of an agent to be tested, and comparing the amount of FRET measured in step (ii) to the amount of FRET measured in step (iv).

91. A screening method for the identification of a pro-apoptotic agent the method comprising
i) providing a granulocyte obtainable from a genetically modified aquatic vertebrate organism, the granulocyte comprising a fusion protein wherein the fusion protein comprises a donor fluorophore and an acceptor fluorophore linked by a peptide comprising a protease cleavage site;
ii) measuring the amount of fluorescence resonance energy transfer (FRET) in the granulocyte in the absence of an agent to be tested;
iii) exposing the granulocyte to an agent to be tested;
iv) measuring the amount of FRET in the granulocyte in the presence of an agent to be tested, wherein if the amount of FRET measured in step (ii) is greater than the amount of FRET measured in step (iv) then the agent is a pro-apoptotic agent.

92. A method as claimed in claim 90 wherein the method includes the steps of:
v) collating the activity data in steps (ii) and (iv);
vi) converting the collated data into a data analysable form; and
vii) optionally providing an output for the analysed data.

93. A method as claimed in 91 wherein the method includes the steps of:
v) collating the activity data in steps (ii) and (iv);
vi) converting the collated data into a data analysable form; and
vii) optionally providing an output for the analysed data.

94. A method as claimed in claim 91 wherein the donor and acceptor fluorophores are green fluorescent proteins or derivatives thereof selected from the group consisting of include blue fluorescent protein (BFP), yellow fluorescent protein (YFP), cyan fluorescent protein (CFP), enhanced cyan yellow protein (ECYP), EYFP and EGFP.

95. A method as claimed in claim 91 wherein the protease is selected from the group consisting of calthains, cathepsins and caspases.

96. A screening method for the identification of agents that induce granulocyte apoptosis the method comprising the steps of
i) providing a granulocyte comprising a nucleic acid molecule comprising
a) a nucleic acid sequence encoding a fusion protein wherein the fusion protein comprises a donor fluorophore and an acceptor fluorophore linked by a peptide comprising a protease cleavage site; and
b) a nucleic acid sequence comprising all, or the regulatory part thereof, of the promoter sequence for a gene encoding a protein expressed in granulocytes operably linked to the nucleic acid in (a);
ii) assessing the amount and distribution of FRET in the granulocyte in the absence of an agent to be tested;
iii) exposing the granulocyte to an agent to be tested;
iv) assessing the amount and distribution of FRET in the granulocyte in the presence of an agent to be tested, wherein if the amount of FRET measured in step (ii) is greater than the amount of FRET measured in step (iv) then the agent is a pro-apoptotic agent.

97. A method as claimed in claim 95 wherein the promoter is a neutrophil specific gene promoter.

98. A method as claimed in claim 96 wherein the neutrophil specific gene is a fish gene and optionally is a zebrafish gene, and the neutrophil specific gene promoter is a myeloperoxidase promoter.

99. A screening method for the identification of agents that induce granulocyte apoptosis the method comprising the steps of
i) providing a preparation comprising a first granulocyte in the absence of an agent to be tested wherein said granulocyte comprises
a) a nucleic acid sequence encoding a fusion protein wherein the fusion protein comprises a donor fluorophore and an acceptor fluorophore linked by a peptide comprising a protease cleavage site; and
b) a nucleic acid sequence comprising all, or the regulatory part thereof, of the promoter sequence for a gene encoding a protein expressed in granulocytes operably linked to the nucleic acid in (a); and
ii) providing a preparation comprising a second granulocyte in the presence of an agent to be tested wherein said granulocyte comprises
a) a nucleic acid sequence encoding a fusion protein wherein the fusion protein comprises a donor fluorophore and an acceptor fluorophore linked by a peptide comprising a protease cleavage site; and
b) a nucleic acid sequence comprising all, or the regulatory part thereof, of the promoter sequence for a gene encoding a protein expressed in granulocytes operably linked to the nucleic acid in (a);
ii) comparing the amount of FRET measured in the first granulocyte with the amount of FRET measured in the second granulocyte wherein if the amount of FRET measured in the first granulocyte is greater than the amount of FRET measured in second granulocyte then the agent is a pro-apoptotic agent.

100. A granulocyte transfected with a nucleic acid molecule wherein the nucleic acid molecule comprises
i) a nucleic acid sequence encoding a fusion protein wherein the fusion protein comprises a donor fluorophore and an acceptor fluorophore linked by a peptide comprising a protease cleavage site; and
ii) a nucleic acid sequence comprising all, or the regulatory part thereof, of the promoter sequence for a gene encoding a protein expressed in granulocytes operably linked to the nucleic acid in (i).

101. A screening method for the identification of a pro-apoptotic agent the method comprising
i) providing a granulocyte derived from a transgenic aquatic vertebrate organism comprising a polypeptide wherein the polypeptide comprises a green fluorescent protein;
ii) measuring the amount of fluorescence in the granulocyte in the absence of an agent to be tested;
iii) exposing the granulocyte to an agent to be tested; and
iv) measuring the amount of fluorescence in the cells in the presence of an agent to be tested, wherein if the amount of fluorescence measured in step (iv) is less than the amount of fluorescence measured in step (ii) then the agent is pro-apoptotic.

102. A screening method for the identification of a pro-apoptotic agent the method comprising the steps of
i) forming a preparation comprising a granulocyte derived from a transgenic aquatic vertebrate organism and an agent to be tested wherein the granulocyte includes a nucleic acid molecule comprising
a) a nucleic acid sequence encoding a green fluorescent protein; and
b) a nucleic acid sequence comprising all, or the regulatory part thereof, of the promoter sequence for a gene encoding a protein expressed in granulocyte operably linked to the nucleic acid in a);
ii) measuring the amount of fluorescence in the granulocyte in the absence of an agent to be tested;
iii) exposing the granulocyte to an agent to be tested; and
iv) measuring the amount of fluorescence in the cells in the presence of an agent to be tested, wherein if the amount of fluorescence measured in step (iv) is less than the amount of fluorescence measured in step (ii) then the agent is pro-apoptotic.
